(19) United States
(12) Patent Application Publication
(10) Pub. No.: US 2026/0165815 A1
(43) Pub. Date: Jun. 18, 2026

(54) GUIDED BONE REGENERATION SURGICAL PROCEDURE METHOD FOR MINIMIZING OPERATIVE AREA AND PREVENTING LOSS OF BONE GRAFT MATERIAL

(71) Applicants: Jong Jin KIM, Seoul (KR); DMAX CO., LTD., Daegu (KR)

(72) Inventors: Tae Seok PARK, Seoul (KR); Jong Jin KIM, Seoul (KR)

(21) Appl. No.: 18/985,075

(22) Filed: Dec. 18, 2024

(51) Int. Cl. A61C 5/68; A61L 27/36

(52) U.S. Cl. CPC A61C 5/68; A61L 27/365; A61L 2430/02

(57) ABSTRACT

Provided is a guided bone regeneration (GBR) surgical procedure method for minimizing an operative area and preventing the loss of bone graft material, which fabricates and temporarily installs a guide with the same shape as the extraction socket through oral scanning and allows a surgical area of the extraction socket to be intuitively marked to minimize the incision and detachment range of the surgical area, installs bone shields made of bone substitute material on the outer and inner surfaces of the extraction socket, from which the granulation tissue is detached, to reinforce the gum's structural integrity weakened by the detachment and preserve bone graft material by actively preventing the loss of bone graft material, and enables the vertical bone grafting procedure, which was relatively difficult to perform, to be more easily implemented using digital cone beam computed tomography (CBCT) and scanning.

### BACKGROUND OF THE INVENTION

### Field of the Invention

The present invention relates to a guided bone regeneration (GBR) surgical procedure method, which can prevent the invasion of other tissues, such as gums, into an extraction socket and promote smooth bone regeneration by installing a bone graft material (Bio-Oss) or a barrier membrane in the extraction socket. More specifically, the present invention relates to a guided bone regeneration (GBR) surgical procedure method for minimizing an operative area and preventing the loss of bone graft material, which fabricates and temporarily installs a guide with the same shape as the extraction socket through oral scanning and allows a surgical area of the extraction socket to be intuitively marked to minimize the incision and detachment range of the surgical area, installs bone shields made of bone substitute material on the outer and inner surfaces of the extraction socket, from which the granulation tissue is detached, to reinforce the gum's structural integrity weakened by the detachment and preserve bone graft material by actively preventing the loss of bone graft material, and enables the vertical bone grafting procedure, which was relatively difficult to perform, to be more easily implemented using digital cone beam computed tomography (CBCT) and scanning, thereby significantly improving the success rate of both vertical and horizontal bone grafting procedures, which were previously limited, enhancing the ease of handling, use, maintenance, and management, and maximizing convenience and efficiency.

### Background Art

In general, when the remaining alveolar bone in the alveolar canal is insufficient for implant placement, guided bone regeneration (GBR) surgical procedure is performed. Here, GBR refers to a surgical procedure in which a barrier membrane is installed in a bone loss area to promote bone regeneration.

The GBR surgical procedure generally consists of tooth extraction, incision and detachment of the surrounding tissue of a extraction socket, placement of the bone graft material, and re-suturing of the detached skin. Depending on the condition of the patient's extraction socket, additional procedures may be included or excluded.

Meanwhile, as a GBR-related technology, Korean Patent No. 10-1260757 discloses “Multilayer absorbable periodontal tissue regeneration membrane”. The technology prevents rapid degradation after transplantation by combining biodegradable materials and hydrogels, and effectively blocks the infiltration of soft tissues into the barrier membrane, thereby guiding the barrier membrane to serve the function during the bone formation period.

The above-mentioned technology applies hydrogels to biodegradable materials for the barrier membrane to alleviate problems such as excessively rapid degradation, weak mechanical strength, and low space maintenance ability due to soft tissue infiltration in a single-layer absorbable barrier membrane. As a result, the multilayer composite absorbable barrier membrane prevents rapid degradation after transplantation and effectively blocks soft tissue infiltration, securing sufficient time for bone formation.

As a GBR-related technology, Korean Patent No. 10-1800538 discloses “Adhesive alveolar bone barrier membrane and a manufacturing method thereof”. The above-mentioned technology includes: a cover part which covers an upper part of an alveolar bone loss area; and an adhesive part which extends from the cover part and adheres to the alveolar bone in an area adjacent to the alveolar bone loss area.

The described technique can actively restore the original shape of the lost alveolar bone by bonding the adhesive part to the alveolar bone using adhesive, and significantly reduce the procedure time compared to the procedures using screws or sutures.

For instance, the conventional GBR surgical procedures often failed to fully restore the original shape of the alveolar bone due to the movement of bone graft material or the barrier membrane during surgery, causing significant inconvenience.

Additionally, in cases where patients leave the extraction socket untreated for a long period of time after extraction, due to the regeneration of the gum over the extraction socket, it is difficult to identify the precise location of the extraction socket. Accordingly, an operator inevitably had to expand the gum detachment range to grasp the location of the extraction socket, leading to prolonged procedure time and extended recovery time for the gums.

Thus, the present invention has been developed to perfectly restore the alveolar bone to the original form by stably maintaining the bone graft material or the barrier membrane during the procedure, clearly identify the location of the extraction socket even in patients who have left the gums untreated for a long period of time after extraction, thus minimizing the surgical range, namely, the detachment range of the granulation tissue.

### PATENT LITERATURE

### Patent Documents

Patent Document 1: Korean Patent No. 10-1260757 entitled “Multilayer absorbable periodontal tissue regeneration membrane”

Patent Document 1: Korean Patent No. 10-1800538 entitled “Adhesive alveolar bone barrier membrane and a manufacturing method thereof”

### SUMMARY OF THE INVENTION

Accordingly, the present invention has been made to solve the above-mentioned problems occurring in the prior arts, and it is an objective of the present invention to provide a technology capable of preventing a bone graft material and a barrier membrane inserted or installed in an extraction socket from being lost or moved due to detachment of the granulation tissue around the extraction socket.

It is another objective of the present invention to provide a technology that can facilitate the precise identification of the location of the extraction socket for GBR surgical procedures, even in cases where patients have left the gums untreated for a long period of time after extraction and the gums have partially regenerated, thus minimizing the incision and detachment range of the granulation tissue.

To accomplish the above objectives, according to the present invention, there is provided a guided bone regeneration (GBR) surgical procedure method for minimizing an operative area and preventing the loss of bone graft material as follows.

The guided bone regeneration (GBR) surgical procedure method of the present invention includes: fabricating a temporary structure with the same shape as a tooth extracted through scanning of a patient's oral cavity; pre-installing the temporary structure in an extraction socket where guided bone regeneration (GBR) will be performed to intuitively guide a surgical area, minimizing the incision and detachment of the gum; after removing the temporary structure, attaching bone shields to the outer and inner sides of the detached gum to actively prevent the loss of bone graft material applied for bone reconstruction.

Here, the temporary structure includes: a guide, which is fabricated to have a shape identical to or corresponding to the shape of the extracted tooth or the empty space of the extraction socket through scanning of a patient's oral cavity, is temporarily inserted into the extraction socket, and then, is removed after the detachment of the granulation tissue; and connectors, which are connected to the top of the guide by screws, extend to the left and right from the guide, and are caught to the unoperated tooth around the extraction socket, contributing to temporary fixing of the guide.

The guided bone regeneration (GBR) surgical procedure method includes: a skin detachment step (S10) of installing the temporary structure in the extraction socket, incising the granulation tissue vertically downward from the top of the extraction socket along the edge of the guide projected to the outside of the gum, and detaching the granulation tissue; an alveolar bone grafting step (S20) of attaching the bone shields to the outer and inner sides of the extraction socket and inserting the bone graft material; and a skin suturing step (S30) of suturing the detached gum tissue over the extraction socket to complete the procedure.

Additionally, the skin detachment step (S10) includes: a gum opening step (S11) of incising the top of the gum to open the extraction socket; a temporary structure installation step (S12) of inserting the temporary structure into the extraction socket to temporarily fix the temporary structure; a gum incision step (S13) of minimally incising the outer and inner sides of the extraction socket along the edge of the guide projected to the gum; and a temporary structure removal step (S14) of removing the temporary structure and detaching the incised skin.

In addition, the alveolar bone grafting step (S20) includes: a bone shield fixing step (S21) of placing the bone shields on the outer and inner sides of the extraction socket and fixing the bone shields to the remaining alveolar bone using screws; and a bone graft material insertion step (S22) of transplanting the bone graft material into the extraction socket formed by the bone shields.

As described above, according to the present invention, the temporary structure inserted into the extraction socket just by simply inserting and installing the temporary structure projects outside the gum or creates a three-dimensional shape on the extraction socket by the temporary structure, allowing the operator to intuitively identify the location of the extraction socket, thus minimizing the incision and detachment range of the granulation tissue around the extraction socket. Thus, the present invention can actively reduce the treatment time and the gum recovery time, so is very effective. Additionally, the present invention prevents the loss of the bone graft material since the bone shields reinforce or shield the gums around the extraction socket which is weakened in the gum's structural integrity or communicates with the outside due to the detachment of the granulation tissue, thus actively contributing to the restoration of the original shape of the alveolar bone.

Furthermore, unlike traditional methods that faced many difficulties in vertical bone grafting due to the inaccuracy of digital cone beam computed tomography (CBCT) and digital scanning, the present invention enhances the accuracy of digital CBCT and scanning based on the local treatment using the temporary structure and the technology of stabilized bone graft material filling using the bone shields, thus improving accessibility to relatively difficult bone graft procedures such as vertical and horizontal grafting, increasing expectations for limited bone grafting in implant procedures, and actively enhancing the success rate of complex bone grafting.

### BRIEF DESCRIPTION OF THE DRAWINGS

FIG. 1 is a schematic diagram illustrating the conventional GBR procedure, which expand the incision and detachment range of the granulation tissue since a location of an extraction socket is not specified.

FIG. 2 is a schematic diagram illustrating the conventional GBR procedure, where the surrounding gum of the extraction socket loses structural integrity, causing the bone graft material to shift.

FIG. 3 is a flowchart illustrating a guided bone regeneration surgical procedure stage by stage according to a preferred embodiment of the present invention.

FIG. 4 is a perspective view of a temporary structure proposed by the present invention.

FIG. 5 is an illustrative diagram showing a patient's gum with a minimized incision and detachment range of the granulation tissue using the temporary structure proposed by the present invention.

FIG. 6 is an illustrative diagram showing an extraction socket where bone graft material has been stably inserted using bone shields proposed by the present invention.

### DETAILED DESCRIPTION OF THE PREFERRED EMBODIMENT

Hereinafter, the structure, functions, and effects of the present invention will be collectively described with reference to the accompanying drawings.

Advantages and features of the present disclosure and methods accomplishing the advantages and features will become apparent from the following detailed description of exemplary embodiments with reference to the accompanying drawings. However, the present invention is not limited to exemplary embodiment disclosed herein but will be implemented in various forms. The exemplary embodiments are provided so that the present invention is completely disclosed, and a person of ordinary skilled in the art can fully understand the scope of the present invention. Therefore, the present invention will be defined only by the scope of the appended claims. Throughout the specification, the same reference numerals denote the same components.

The present invention relates to a guided bone regeneration (GBR) surgical procedure method, which can prevent the invasion of other tissues, such as gums, into an extraction socket and promote smooth bone regeneration by installing a bone graft material (Bio-Oss) or a barrier membrane in the extraction socket.

Above all, the present invention relates to a guided bone regeneration (GBR) surgical procedure method for minimizing an operative area and preventing the loss of bone graft material, which fabricates and temporarily installs a guide with the same shape as the extraction socket through oral scanning and allows a surgical area of the extraction socket to be intuitively marked to minimize the incision and detachment range of the surgical area, installs bone shields made of bone substitute material on the outer and inner surfaces of the extraction socket, from which the granulation tissue is detached, to reinforce the gum's structural integrity weakened by the detachment and preserve bone graft material by actively preventing the loss of bone graft material, and enables the vertical bone grafting procedure, which was relatively difficult to perform, to be more easily implemented using digital cone beam computed tomography (CBCT) and scanning, thereby significantly improving the success rate of both vertical and horizontal bone grafting procedures, which were previously limited, enhancing the ease of handling, use, maintenance, and management, and maximizing convenience and efficiency.

The previously challenging vertical bone grafting procedure can now be implemented more easily using digital CBCT and scanning. Consequently, the invention significantly improves the success rate of both vertical and horizontal bone grafting procedures, which were previously limited. The innovative structure also ensures ease of handling, use, maintenance, and repair, ultimately maximizing convenience and efficiency. The invention pertains to a GBR surgical method that minimizes the surgical area and prevents the loss of bone graft material.

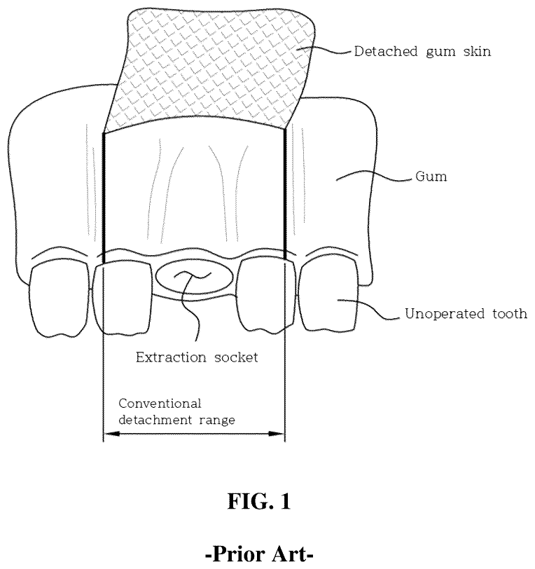

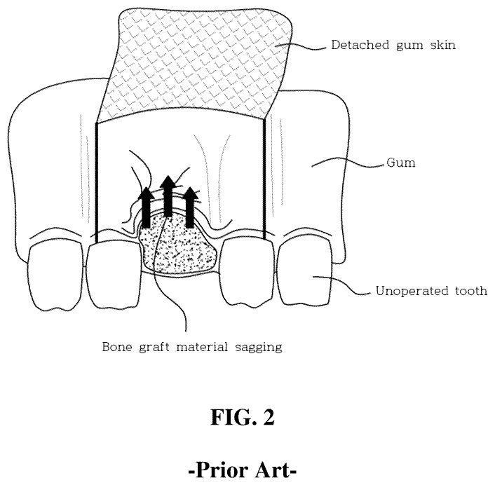

FIG. 1 is a schematic diagram illustrating the conventional GBR procedure, which expand the incision and detachment range of the granulation tissue since a location of an extraction socket is not specified, and FIG. 2 is a schematic diagram illustrating the conventional GBR procedure, where the surrounding gum of the extraction socket loses structural integrity, causing the bone graft material to shift.

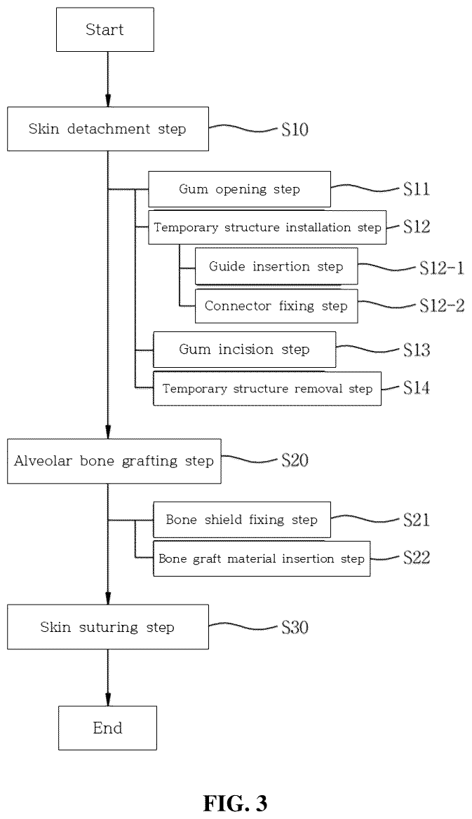

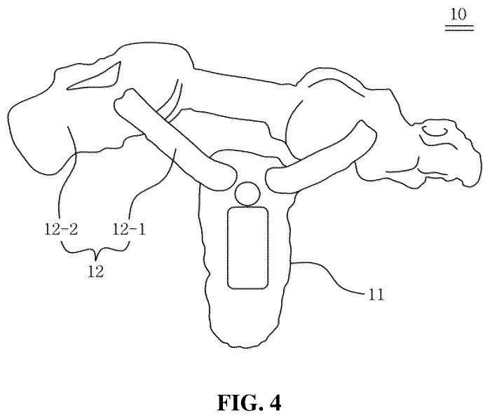

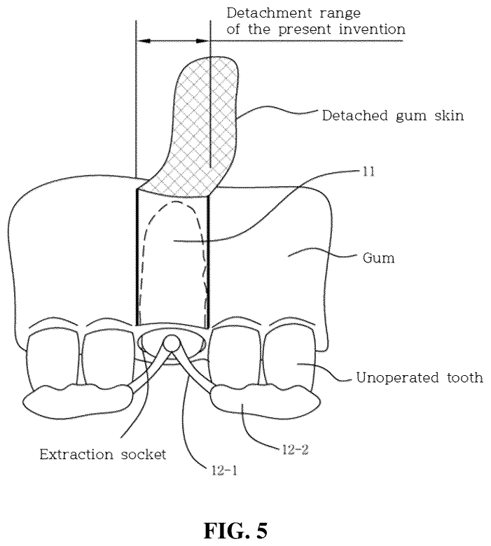

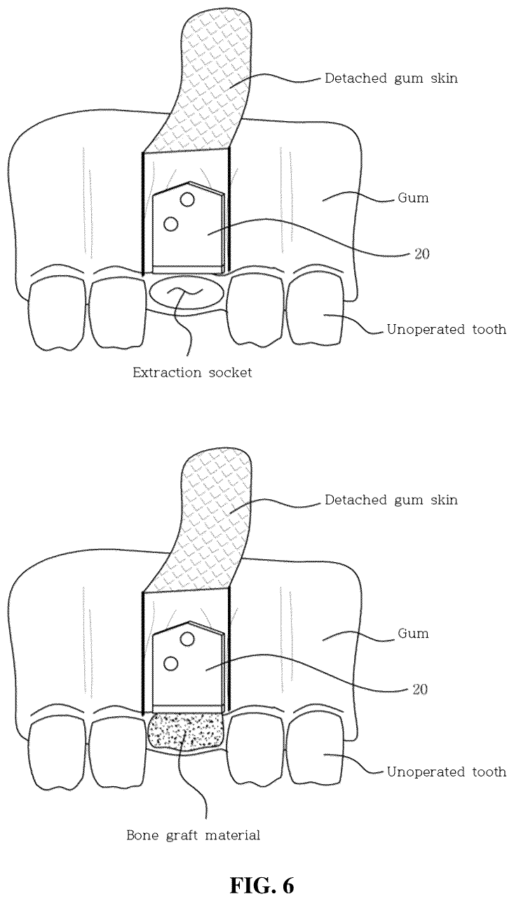

FIG. 3 is a flowchart illustrating a guided bone regeneration surgical procedure stage by stage according to a preferred embodiment of the present invention, FIG. 4 is a perspective view of a temporary structure proposed by the present invention, FIG. 5 is an illustrative diagram showing a patient's gum with a minimized incision and detachment range of the granulation tissue using the temporary structure proposed by the present invention, and FIG. 6 is an illustrative diagram showing an extraction socket where bone graft material has been stably inserted using bone shields proposed by the present invention.

As illustrated in FIG. 1, in cases where patients leave the extraction socket untreated for a long period of time after extraction, due to the regeneration of the gum over the extraction socket makes, the conventional GBR surgical procedures were difficult to identify the precise location of the extraction socket, so an operator inevitably had to expand the gum detachment range to grasp the location of the extraction socket, leading to prolonged procedure time and extended recovery time for the gums.

Additionally, as illustrated in FIG. 2, the conventional GBR surgical procedures deteriorated the gum's structural integrity due to the detachment of the granulation tissue surrounding the extraction socket and failed to fully restore the original shape of the alveolar bone due to the movement of bone graft material or the barrier membrane during surgery, resulting in reduced procedural completeness and longer surgical times.

Therefore, an objective of the present invention is to propose a technology that can perfectly restore the alveolar bone to the original form by positively preventing the loss of the bone graft material during the GBR surgical procedure, and clearly identify the location of the extraction socket even in patients who have left the gums untreated for a long period of time after extraction, thereby minimizing the detachment of the granulation tissue.

As illustrated in FIG. 3, the GBR method proposed by the present invention includes: fabricating a temporary structure 10 with the same shape as a tooth extracted through scanning of a patient's oral cavity; pre-installing the temporary structure in an extraction socket where guided bone regeneration (GBR) will be performed to intuitively guide a surgical area, minimizing the incision and detachment of the gum; after removing the temporary structure, attaching bone shields 20 to the outer and inner sides of the detached gum to actively prevent the loss of bone graft material applied for bone reconstruction.

The GBR procedure includes: a skin detachment step (S10) of installing the temporary structure 10 in the extraction socket and incising and detaching the granulation tissue referring to the temporary structure projecting to the outside of the gum; an alveolar bone grafting step (S20) of attaching the bone shields 20 to the outer and inner sides of the extraction socket and inserting the bone graft material; and a skin suturing step S30 of suturing the detached gum tissue over the extraction socket to complete the procedure.

The skin detachment step (S10) includes: a gum opening step (S11) of opening the extraction socket; a temporary structure installation step (S12) of installing the temporary structure 10 in the extraction socket; a gum incision step (S13) of incising the gum on the outer and inner sides of the extraction socket; and a temporary structure removal step (S14) of removing the temporary structure after detaching the granulation tissue.

The gum opening step (S11) involves securing the extraction socket from the extracted gum. Specifically, the gum opening step (S11) is performed by different surgery methods depending on a case where the location of the extraction socket can be identified before gum regeneration after the detachment and a case where the location of the extraction socket cannot be identified due to the regeneration of some of the gum over the extraction socket untreated for a long period of time after extraction. Here, in the former case, the gum opening step (S11) includes only a process of opening the extraction socket using a medical tool or removing blood and debris cumulated on the extraction socket using a suction. However, in the latter case, the gum opening step (S11) includes incising the upper surface of the gum to make the extraction socket communicate with the outside; and opening the extraction socket using a medical tool or removing blood and debris cumulated on the extraction socket using a suction.

The temporary structure installation step (S12) is to install the temporary structure 10 in the extraction socket. Here, the temporary structure 10 includes a guide 11 shaped to match an empty space of the extraction socket; and connectors 12 extending to both sides from the top of the guide. The temporary structure installation step (S12) of installing the temporary structure 10 having the above configuration in the extraction socket includes: a guide insertion step (S12-1) of temporarily inserting the guide 11 into the extraction socket; and a connector fixing step (S12-2) of pulling the connectors 12 extended from the guide to the right and left and fixing the connectors to unoperated teeth around the extraction socket.

The installation of the temporary structure 10 in the extraction socket ensures that the guide 11 projects externally from the gum or creates a three-dimensional shape on the extraction socket by the temporary structure, allowing an operator to intuitively identify the location and range of the extraction socket, thus minimizing the detachment range of the granulation tissue in the gum.

The gum incision step (S13) is to incise the gum on the outer and inner sides of the extraction socket, to which the GBR procedure will be performed, based on the shape of the temporary structure 10 projected to the gum. The operator makes a vertical incision downward from the top of the gum using a scalpel, following the shape of the temporary structure 10 installed in the extraction socket.

The temporary structure removal step (S14) involves two actions: removing the temporary structure 10 from the extraction socket after the granulation tissue has been incised; detaching and spreading the incised granulation tissue outward to prepare for bone grafting.

The alveolar bone grafting step S20 of attaching the bone shields 20 to both the outer and inner sides of the gum around the extraction socket and inserting bone graft material includes: a bone shield fixing step S21 of placing the bone shields 20 on the outer and inner sides of the extraction socket and fixing the bone shields to the remaining alveolar bone using screws; and a bone graft material insertion step (S22) of transplanting the bone graft material into the extraction socket formed by the bone shields. Here, the bone shields 20 are pieces of bone, designed to have a width and a shape to cover a reinforced area of the extraction socket from which the granulation tissue was detached. The bone shields 20 can be actual bone fragments or artificial bone pieces. The bone shields 20 may have the shape illustrated in FIG. 6, or may vary depending on the size and shape of the reinforced area.

The bone shield fixing step (S21) can include: a step of placing the bone fragment on the reinforced area of the extraction socket; and a step of penetrating the bone fragment with screws and fixing the bone fragment to the alveolar bone remaining within the gum.

The bone graft material insertion step (S22) involves inserting the bone graft material into the extraction socket after the area around the extraction socket is stabilized by the bone shields 20. The bone graft material can be bone powder or Bio-Oss. For instance, if the bone graft material is bone powder, the bone graft material can be filled into the socket immediately. However, if the bone graft material is Bio-Oss, cleaning work may be required.

If the bone graft material is Bio-Oss, the bone graft material insertion step (S22) includes: a step of soaking Bio-Oss in saline solution; a step of thawing Regenaform; a step of transplanting Bio-Oss and Regenaform into the extraction socket; and a step of overlapping a membrane along the surface of the bone graft material.

The tissue suturing step S30 includes: a step of covering the barrier membrane with the detached granulation tissue; a step of suturing the incision area; a step of allowing time for the regeneration of the alveolar bone and healing of the gum; and a step of lifting a flap and removing the barrier membrane.

In the tissue suturing step S30, after the barrier membrane is removed, an implant procedure is immediately performed.

Meanwhile, the temporary structure 10 of the present invention may include: a guide 11, which is fabricated to have a shape identical to or corresponding to the shape of the extracted tooth or the empty space of the extraction socket through scanning of a patient's oral cavity, is temporarily inserted into the extraction socket, and then, is removed after the detachment of the granulation tissue; and connectors 12, which are connected to the top of the guide by screws, extend to the left and right from the guide, and are caught to the unoperated tooth around the extraction socket, contributing to temporary fixing of the guide.

The guide 11 is manufactured using scanning of a patient's oral cavity and 3D printing, and corresponds to the root area of the tooth. The guide 11 can obtain the outward shape from the tooth before extraction or obtain the outward shape by scanning the empty space of the extraction socket formed in the gum after extraction.

For example, as the guide 11 inserted into the extraction socket slightly projects externally, the operator can intuitively identify the area of the extraction socket through the projecting guide. So, it guides the incision of the granulation tissue along the shape of the guide, enabling a minimally invasive procedure rather than the conventional wide-scale incision. As a result, the surgical area is reduced, thus significantly shortening the difficulty and duration of the procedure and reducing the patient's recovery time.

The connector 12 can be manufactured depending on the teeth arrangement conditions through the scanning of a patient's oral cavity, and includes: an extension part 12-1 having the length determined by the distance between the extraction socket and the nearest unoperated tooth; and a fixing part 12-2 encircling a portion of the top of the unoperated tooth and having a shape corresponding to the convex and concave contours of the unoperated tooth.

The connector 12 may include a structure of being assembled integrally with the guide 11 via screws to be detachably attached to the guide.

According to the present invention, the temporary structure inserted into the extraction socket just by simply inserting and installing the temporary structure projects outside the gum or creates a three-dimensional shape on the extraction socket by the temporary structure, allowing the operator to intuitively identify the location of the extraction socket, thus minimizing the incision and detachment range of the granulation tissue around the extraction socket. Thus, the present invention can actively reduce the treatment time and the gum recovery time, so is very effective. Additionally, the present invention prevents the loss of the bone graft material since the bone shields reinforce or shield the gums around the extraction socket which is weakened in the gum's structural integrity or communicates with the outside due to the detachment of the granulation tissue, thus actively contributing to the restoration of the original shape of the alveolar bone.

Furthermore, unlike traditional methods that faced many difficulties in vertical bone grafting due to the inaccuracy of digital cone beam computed tomography (CBCT) and digital scanning, the present invention enhances the accuracy of digital CBCT and scanning based on the local treatment using the temporary structure and the technology of stabilized bone graft material filling using the bone shields, thus improving accessibility to relatively difficult bone graft procedures such as vertical and horizontal grafting, increasing expectations for limited bone grafting in implant procedures, and actively enhancing the success rate of complex bone grafting.

As previously described, in the detailed description of the invention, having described the detailed exemplary embodiments of the invention, it should be apparent that modifications and variations can be made by persons skilled without deviating from the spirit or scope of the invention. Therefore, the true technical scope of the present invention should be interpreted based on the appended claims, and all technical ideas within an equivalent scope should be construed as being included in the scope of the present invention.

1. A guided bone regeneration (GBR) surgical procedure method, comprising:
scanning a patient's oral cavity to fabricate a temporary structure having a shape identical to or corresponding to a shape of an extracted tooth;
inserting the temporary structure into an extraction socket to identify a gum incision area, wherein the inserting of the temporary structure enables identification of the gum incision area by visually confirming a location and range of the extraction socket;
making a gum incision along the identified gum incision area;
removing the temporary structure from the extraction socket;
attaching bone shields to outer and inner sides of the extraction socket;
inserting a bone graft material into an empty space formed by removing the temporary structure from the extraction socket; and
suturing the incision area.

2. The guided bone regeneration (GBR) surgical procedure method according to claim 1, wherein the temporary structure comprises:
a guide, which is fabricated to have the shape identical to or corresponding to the shape of the extracted tooth or the empty space of the extraction socket through the scanning of the patient's oral cavity; and
connectors, which are connected to a top of the guide by screws, extend to left and right from the guide, and are caught to an unoperated tooth around the extraction socket.

3. The guided bone regeneration (GBR) surgical procedure method according to claim 1, comprising:
a granulation tissue detachment step (S10) including making the gum incision along the identified gum incision area and detaching the granulation tissue from a top of the extraction socket;
an alveolar bone grafting step (S20) of attaching the bone shields to the outer and inner sides of the extraction socket and inserting the bone graft material; and
a suturing step (S30) of suturing the detached granulation tissue over the extraction socket.

4. The guided bone regeneration (GBR) surgical procedure method according to claim 3, wherein the granulation tissue detachment step (S10) comprises:
a gum opening step (S11) of incising the top of the gum to open the extraction socket;
a temporary structure installation step (S12) of inserting the temporary structure into the extraction socket to temporarily fix the temporary structure;
a gum incision step (S13) of incising the outer and inner sides of the extraction socket along the identified gum incision area; and
a temporary structure removal step (S14) of removing the temporary structure after detaching the incised granulation tissue.

5. The guided bone regeneration (GBR) surgical procedure method according to claim 3, wherein the alveolar bone grafting step (S20) comprises:
a bone shield fixing step (S21) of placing the bone shields on the outer and inner sides of the extraction socket; and
a bone graft material insertion step (S22) of transplanting the bone graft material into the extraction socket defined by the bone shields.
